(19) United States
(12) Patent Application Publication
(10) Pub. No.: US 2014/0213775 A1
(43) Pub. Date: Jul. 31, 2014

(54) INTRACELLULAR CELL SELECTION

(71) Applicant: Abeterno Limited, Cambridge (GB)

(72) Inventor: Rosalind Codrington, Impington (GB)

(21) Appl. No.: 14/241,215

(22) PCT Filed: Sep. 11, 2012
(86) PCT No.: PCT/GB2012/052227

§ 371 Date: Feb. 26, 2014

(30) Foreign Application Priority Data
Sep. 14, 2011 (GB) 1115847.4
Apr. 24, 2012 (GB) 1207177.5

(51) Int. Cl. C07K 17/00

(52) U.S. Cl. CPC C07K 17/00
USPC 530/391.7

(57) ABSTRACT

The disclosure relates to a magnetic particle comprising: a ligand that specifically binds an intracellular antigen, a cell membrane penetrating cationic peptide and a peptide comprising an amino acid sequence that is adapted to interact with the secretory pathway in a cell and the use of the particle in selection of cells from heterogeneous cell populations.

### INTRODUCTION

This disclosure relates to the selection and isolation of mammalian cells from a heterogeneous population of mammalian cells by affinity binding of intracellular antigens by ligands, typically antibody fragments, and the use of magnetic particles to select said cells.

### BACKGROUND

Selective cell separation is an essential method in experimental biology and medicine. It is an increasingly important process that is driven by the ever increasing demands for sensitivity, selectivity, yield, time and economy of the process. The most popular aim for cell separation is to achieve complete removal of the target population from a cell mixture. However, for some applications, a secondary aim may be to recover the selected cells for study for additional manipulation. Additionally, the purity of separation may be more important than the yield of the selected cells, or vice versa. Thus, the aims for cell separation determine the method that is chosen to achieve the separation. Other factors to take into account when selecting the separation technique are the processing time, viability of residual cells, and exposure to non-medically approved reagents.

There are a number of cell separation techniques which can be broadly classified into two categories: techniques based on size and density, and techniques based on affinity (chemical, electrical, or magnetic) (Radisic et al, 2006).

Magnetic separation techniques are a common method in many laboratories and have been broadly used for targeted drug delivery, imaging and bioseparation such as in selective cell separations, antibody purification, protein affinity purification, immunoprecipitations, protein fractionations, organelle isolations, total nucleic acid purification and polyadenylated mRNA purification (Saiyed et al, 2003; Nord et al, 2001; Stark et al, 1988; Suzuki et al, 1996; Weissleder et al, 1997).

The magnetic cell separation technique consists of magnetic beads coated with antibodies specific for cell surface antigens on the desired cells. When the antibody-magnetic beads are exposed to a mixed cell population, they attach to the surface of the desired cells via antibody-antigen interaction. The desired cell subpopulation can then be separated in the presence of a strong magnetic field. The antibodies used in these applications are typically monoclonal antibodies specific for cell surface antigens. However, antibody fragments such as single chain antibodies (scFv) have also been successfully coupled to magnetic beads and used in a variety of applications such as phage display libraries of scFv and in imaging of tumours (McConnell et al, 1999; Nord et al, 2001; Han et al, 2006).

Single VH and VL domains are the smallest functional modules of antibodies required for antigen binding. ScFv are a fusion of the variable regions of the heavy (VH) and light chains (VL) of immunoglobulins, connected with a short linker peptide of 10 to 25 amino acids. Their small size conveys them with distinct advantages over whole antibodies, in particular they have rapid pharmacokinetics, greater tumour penetration and lower immunogenicity than intact IgG, F(ab′)₂, or Fab (Yokota et al., 1992). They have also shown to be particularly useful in clinical applications involving the selective delivery of radionucleotides to tumours (Huston et al., 1996; Reisfeld et al., 1996; Laske et al., 1997). The scFv may be assembled from the variable regions of particular monoclonal antibodies (Huston et al., 1988; Brinkmann et al., 1991) or made de novo from phage display libraries (Winter et al., 1994; Chester et al., 1994). They retain the specificity of the original immunoglobulin, and in many applications outperform whole IgG in construction speed, production yield, and engineering flexibility, as they can be easily expressed in bacterial expression systems. Indeed, many scFv have been produced in bacteria with good yields, either as insoluble inclusion bodies in Escherichia coli (Huston et al., 1988) or by secretion into the periplasm (Skerra et al., 1993) or into the culture supernatant (Takkinen et al., 1991).

A major caveat of magnetic cell separation is that it is only applicable to cells that can be separated by their specific surface antigens. This can be particularly limiting when the cell specific markers are mainly found intracellularly and are not readily available on the cell surface. Indeed, in most cancers, the cancer markers of interest are predominantly expressed inside the cells and therefore cannot be used for cell separations. The inherent difficulties surrounding cell internalisation of antibodies have made it difficult so far to use intracellularly expressed antigens in cell separations. Whole monoclonal antibodies cannot be internalised or be expressed in cells due to the reducing environment of the cell.

However, scFv are routinely stably expressed in cells and have been reportedly been able to internalise into cells with the help of carrier mediated systems such as transduction peptides (Futaki et al, 2001; Avignolo et al, 2008; Niesner et al, 2002; Ma et al, 2011). In addition, recent studies using magnetic particles have shown that it is possible to internalise these particles into cells and control their location by means of an external magnet (Tseng et al., 2009). Moreover, they have been used in cancer therapeutics as a means to direct the cells containing anti-cancer agents to a desired location on the cancer cells by means of an external magnetic field, thus, promising to reduce the cytotoxic effects of drugs by increasing the bioavailability of therapeutic compounds at the site of action and improving pharmacokinetics (Cinti et al., 2011). Magnetic beads have also been used as a means to internalise nucleic acids (magnetofection) (Scherer et al., 2002) and small peptides into cells (Han et al., 2006) and have been previously coupled to cationic peptides (Smith et al., 2002) to enhance their uptake in vivo and allow their use for MRI scanning (Stark et al., 1988; Suzuki et al., 1996; Weissleder et al., 1997). Additionally they have been coupled to drugs for magnetic drug targeting in patients with solid tumours (Lubbe et al., 1996; Alexiou et al., 2000). Furthermore, these magnetic particles do not rely on receptors or other cell membrane bound proteins for their cellular uptake, it is possible to transfect cells that are normally non-permissive (Scherer et al., 2002). This is also true in primary cells which are known to be difficult to transfect (Krotz et al, 2003).

### STATEMENTS OF INVENTION

The present disclosure is directed to overcoming the deficiencies in magnetic cell separation by providing a method to separate a target population from a cell mixture on the basis of the expression of their intracellular markers and to then allow the recovery of these cells for additional downstream studies and to achieve therapeutic benefits.

This disclosure relates to a method for isolating specific cells from a mixed population of different cell types using intracellular markers rather than cell surface markers. This method involves the coupling of antibody fragments to a magnetic particle, where the antibody fragment is specific to the marker of interest expressed inside the cell.

According to an aspect of the invention there is provided a magnetic particle comprising: a ligand that specifically binds an intracellular antigen, a cell membrane penetrating cationic peptide and a peptide comprising an amino acid sequence that is adapted to interact with the secretory pathway in a cell.

In a preferred embodiment of the invention said intracellular antigen is substantially a nuclear antigen.

In a preferred embodiment of the invention said nuclear antigen is selected from the group consisting of: FoxO family members, TARP, p53, Rb, E2F, hK4, BRCA1, Cdk2, SATB1, TP53INP1, Myc, Fos, Jun, CREB, Ets, SRF, FAK, Pax6, Calpain, Elk1, Stats 1-3, Akt, p21

In an alternative preferred embodiment of the invention said intracellular antigen is substantially a cytoplasmic antigen.

In a preferred embodiment of the invention said cytoplasmic antigen is selected from the group consisting of: Jak1, Jak2, Tyk2, Jak3, GATA 1-4, Stats 1-6, CBP, NFkB, IKK, PIK3CA, B-raf, EBI3, eIF2α, Akt, PI3K, IAP, Hsp, FAK, Raf, Ras, TNF, Src, Abl, Caspases 1-12.

In a preferred embodiment of the invention said cationic cell membrane penetrating peptide is a natural cell penetrating peptide.

In an alternative preferred embodiment of the invention said cell membrane penetrating peptide is a synthetic sequence.

In a preferred embodiment of the invention said cell membrane penetrating peptide is adapted to penetrate a mammalian cell.

In a preferred embodiment of the invention said cell membrane penetrating peptide is selected from the group consisting of: YARKKRRQRRR, YARKARRQARR, YARAAARQARA, YARAARRAARR, YARAARRAARA, YARRRRRRRRR, YAAARRRRRRR, PLSSIFSRIGDP, RQIKRVFQNRRMKWKK, WEIEDEDER, GRKKRRQRRRPQ, RRRRRRRRRRRR.

In a preferred embodiment of the invention said cell membrane penetrating peptide consists essentially of the amino acid sequence: GRKKRRQRRRPQ.

Preferably said cell membrane penetrating peptide consists of the amino acid sequence: GRKKRRQRRRPQ.

In a preferred embodiment of the invention said cell membrane penetrating peptide consists essentially of the amino acid sequence: RRRRRRRRRRRR.

Preferably said cell membrane penetrating peptide consists of the amino acid sequence: RRRRRRRRRRRR.

The cell penetrating peptide and the secretory peptide can be engrafted into the CDR3 regions of the VL and VH domains.

In a preferred embodiment of the invention said peptide adapted to interact with the mammalian secretory pathway is selected from the group consisting of: MCPARSLLLVATLVLLDHLSLA, MDAMKRGLCCVLLLCGAVFVSPS, MATGSRTSLL LAFGLLCLPWLQEGSA, MYRMQLLSCIALSLALVTNS or MGVKVLFALICIAVAE.

In a preferred embodiment of the invention said peptide is consists essentially of the amino acid sequence MGVKVLFALICIAVAE.

In a preferred embodiment of the invention said peptide is consists of the amino acid sequence: MGVKVLFALICIAVAE.

In a preferred embodiment of the invention said peptide is consists essentially of the amino acid sequence: MYRMQLLSCIALSLALVTNS.

In a preferred embodiment of the invention said peptide is consists of the amino acid sequence: MYRMQLLSCIALSLALVTNS.

In a preferred embodiment of the invention said ligand that binds said intracellular antigen is an antibody fragment.

Various fragments of antibodies are known in the art, e.g. Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as William E. Paul, Fundamental Immunology or Janeway et al. Immunobiology (cited above). Molecular biology now allows direct synthesis (via expression in cells or chemically) of these fragments, as well as synthesis of combinations thereof. A fragment of an antibody or immunoglobulin can also have bispecific function as described above.

Methods to deliver antibody fragments to cells intracellularly are known in the art; for example see WO2007/064727; WO2004/030610; WO03/095641; WO02/07671; WO01/43778; WO96/40248; and WO94/01131 each of which is incorporated by reference in their entirety.

In a preferred embodiment of the invention said antibody fragment is an ScFv or a single VH or VL domain.

In an alternative preferred embodiment of the invention said ligand that binds said intracellular antigen is a peptide or a peptide aptamer.

Peptides that have binding affinity to a target antigen are within the scope of the invention. For example peptides that mimic the binding affinity of antibodies and antibody fragments; see Chattophadayay, A, Tate, S., Beswick, R., Wagner, S. D. and Ko Ferrigno, P. A peptide aptamer to antagonise BCL-6 function. Oncogene, 25 2223-2233 (2006).

In a preferred embodiment of the invention said cell is a mammalian cell.

In an alternative preferred embodiment of the invention said magnetic particle further comprises a nuclear export peptide.

In a preferred embodiment of the invention said nuclear export peptide comprises or consists essentially of the amino acid sequence: NELALKLAGLDINKTEGEEDAQ

According to a further aspect of the invention there is provided a method for the selection and isolation of a mammalian cell from a heterogeneous population of cells comprising:
i) forming a first preparation comprising a heterogeneous population of mammalian cells;
ii) contacting said population with a second preparation comprising a magnetic particle according to the invention to provide a combined preparation and providing conditions that allow penetration and binding of said magnetic particle to an intracellular antigen;
iii) incubating said combined preparation to remove unbound magnetic particles by the secretory pathway of said mammalian cell, optionally by application of a magnetic field; and
iv) applying a magnetic field to said combined preparation to select and isolate mammalian cells comprising magnetic particles bound to an intracellular antigen.

In a preferred method of the invention said mammalian cell is a stem cell.

Preferably said stem cell is a cancer stem cell.

The concept of a cancer stem cell within a more differentiated tumour mass, as an aberrant form of normal differentiation, is now gaining acceptance over the current stochastic model of oncogenesis, in which all tumour cells are equivalent both in growth and tumour-initiating capacity (Hamburger et al., 1977; Pardal et al., 2003). For example, in leukaemia, the ability to initiate new tumour growth resides in a rare phenotypically distinct subset of tumour cells (Bonnet et al., 1997) which are defined by the expression of CD34+CD38 surface antigens and have been termed leukemic stem cells (LSC). Similar tumour-initiating cells have also been found in ‘solid’ cancers such as prostate (Collins et al., 2005), breast (AlHajj et al., 2003), brain (Singh et al, 2004), lung (Kim et al., 2005), colon (O'Brien et al, 2007; Ricci Vitiani et al., 2007) and gastric cancers (Houghton et al., 2004). A list of intracellular cancer stem cell markers is available in Table 1.

According to an aspect of the invention there is provided a kit for customized modification of the magnetic particles containing a selection of cell membrane penetrating cationic peptides selected from the group of YARKKRRQRRR, YARKARRQARR, YARAAARQARA, YARAARRAARR, YARAARRAARA, YARRRRRRRRR, YAAARRRRRRR, PLSSIFSRIGDP, RQIKIWTQNRRMKWKK, WEIEDEDER, GRKKRRQRRRPQ, RRRRRRRRRRRR, a selection of secretory proteins selected from the group MCPARSLLLVATLVLLDHLSLA, MDAMKRGLCCVLLLCGAVFVSPS, MATGSRTSLLLAFGLLCLPWLQEGSA, MYRMQLLSCIALSLALVTNS or MGVKVLFALICIAVAE and an activation, wash and blocking/storage buffer.

In a preferred embodiment of the invention said kit further comprises a magnet for the use of said customized magnetic particles.

The magnetic particles may be coupled to an antibody fragment (scFv, single domain, dibody), a cell penetrating peptide and a secretory peptide, or to an antibody fragment and a secretory peptide.

The cell penetrating peptide and the secretory peptide may be either directly coupled to the magnetic particle or to the antibody fragment via expression as a fusion protein or via chemical coupling between the antibody and the peptide, or they may be engrafted within the CDR3 regions of the VH or VL domains as previously described by (Jeong et al. 2011).

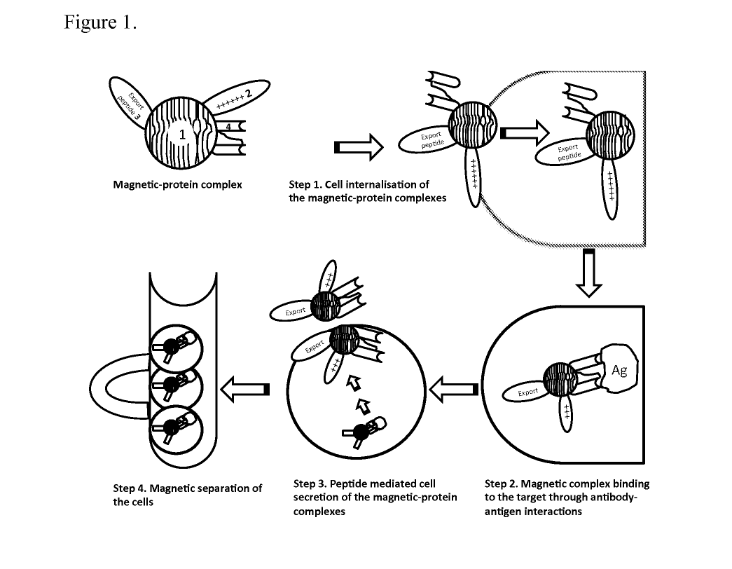

The magnetic antibody fragments are incubated with the cells and allowed to internalize into the cells using the cationic peptide or the positive charge of the magnetic particle as the delivery vehicle. After achieving cell internalisation of the antibody magnetic particles, the cells are allowed a period of incubation in cell media to allow the magnetic antibody fragments bind to their targets. Any unbound magnetic antibody fragments will then be secreted from the cells by the secretory peptide with or without the help of an external magnet. The cells containing just the bound antibody fragments to their respective targets can be selected with the use of an external magnet where any unbound cells are washed away. The selected cells can then be further incubated for other downstream applications (FIG. 1).

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

TABLE 1
Intracellular Cancer stem cell markers

| | | |
|---|---|---|
| TP53INP1 | tumor protein p53 inducible nuclear | ENSG00000164938 |
| | protein 1 | |
| Axl | AXL receptor tyrosine kinase | ENSG00000167601 |
| GATA-3 | GATA binding protein 3 | ENSG00000107485 |
| USP22 | ubiquitin specific peptidase 22 | ENSG00000124422 |
| BIRC6 | baculoviral IAP repeat containing 6 | ENSG00000115760 |
| Hh | sonic hedgehog | ENSG00000164690 |

FIG. 1. Diagram depicting the sequence of events in the magnetic intracellular cell separation technique (MICELS). A magnetic particle (1) is coupled to a cell penetrating positively charged peptide (2), a cell export peptide (3) and an scFv antibody fragment (4). Step 1. The magnetic-protein complex is incubated with the mixed cell population for a length of time until the complex has fully internalised into the cells. Step 2. The scFv antibody fragments specifically bind to their antigen targets (Ag) available in the cells and are thereby anchored inside these cells. Step 3. Any magnetic-protein complexes that are not bound to the specific target antigens are secreted to the extracellular milieu via the export peptide through the cell secretory pathway. This step avoids the selection of cells that do not express the specific target antigens. Step 4. An external magnetic field is applied to the cells allowing only the cells containing the magnetic complexes to bind to the magnet. The remainder of the cell population can be washed away. The selected cells can be now used for further downstream applications.

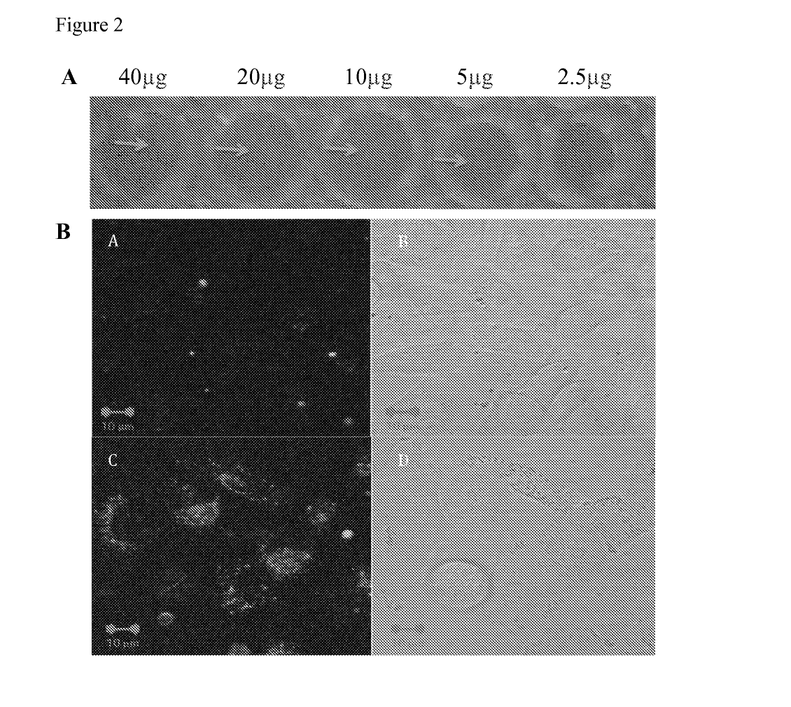

FIG. 2. MICELS selection of CHO and A549 cells with scFv magnetic-protein complexes. (A) MICELS selection of A549 cells in a 24 well plate with a serial dilution of the scFv magnetic-protein complexes (40 μg-2.5 μg). The blue arrows indicate the location of the selected cell pellets. Decreasing amounts of selected cells were obtained with decreasing concentrations of magnetic-protein complexes. The best results for cell selection were obtained with 40 μg of the magnetic-protein complexes. (B) Confocal images of CHO (A, B) and A549 (C, D) cells after 4.30 h incubation with scFv magnetic-protein complexes. The cells were washed vigorously with PBS before imaging. The green magnetic-protein complexes localised at the cell membrane, the perinuclear membrane and in the cytoplasm consistent with the sub-cellular localisation of activated Ras in the A549 lung cancer cell line. No magnetic-protein complexes were observed in CHO cells following 4.30 h incubation.

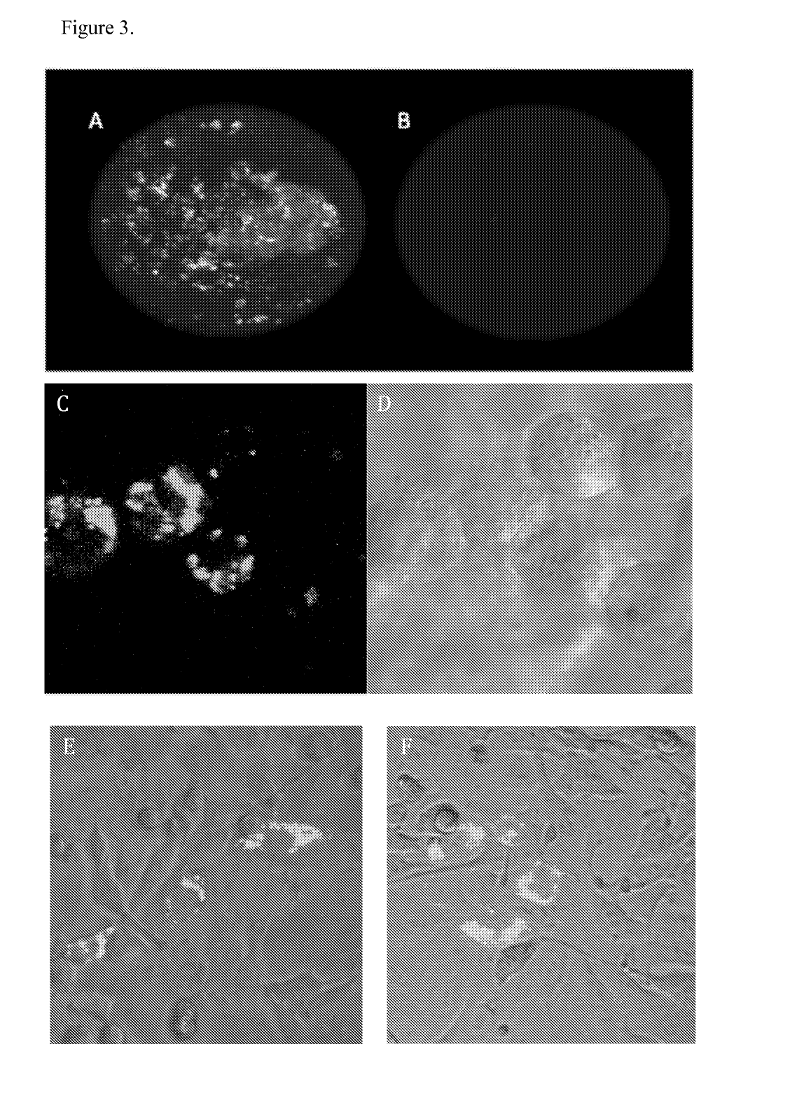

FIG. 3. MICELS selection of A549 and CHO cells with 40 μg scFv magnetic-protein. Images of A549 cells (A) and CHO cells (B) selected using MICELS were imaged using a 10× objective. The images were taken after an extensive wash with PBS followed by trypsinisation and cell selection with the magnet. Minimal background of CHO cells was observed following selection. (C,D) Fluorescent and bright field confocal image of the A549cell pellet obtained after selection. (E,F) Merged confocal images of A549 cells that had been placed back into culture following MICELS selection. The images were taken at 24 h and no significant cytotoxicity was observed.

### MATERIALS AND METHODS

Production of H-RasG12V scFv

The sequence of an scFv (scFv#6) specific for the mutant H-RasG12V (Tanaka et al., EMBO Journal 2007) served as a template for the synthesis of scFv6 by GeneArt(r) (Life Technologies) with flanking BamH1 and EcoR1 sites at the 5′ and 3′ ends, respectively. The gene was subcloned into BamH1 and EcoR1 sites of the pRSETa bacterial expression vector (Life Technologies) to generate in-frame fusion scFv protein with a 6× histidine tag. The plasmid was transformed into BL21 (DE3) bacterial ceils (Merck-Millipore) and expression of the scFv#6 fragment was induced in 500 ml culture via the addition of 1 mM IPTG at OD₆₀₀ of 0.55 followed by incubation for 3 h at 37° C. The cells were harvested by centrifugation at 5000×g at 4° C. and the final cell pellets were stored at −80° C. prior to scFv purification.

scFv protein was extracted from bacterial pellets in 25 ml of B-PER bacterial lysis buffer (Thermo Scientific) in the presence of a cocktail of protease inhibitors (containing 1.5 μg/ml Chymotrypsin, 0.8 μg/ml Thermolysin, 1 mg/ml Papain, 1.5 μg/ml Pronase, 1.5 μg/ml Pancreatic extract, 0.002 μg/ml Trypsin) (Roche) at RT for 30 min. The lysate was centrifuged at 15000×g at 4° C. and the supernatant was diluted in 25 ml lysis buffer (50 mM Na phosphate, pH 8.5, 300 mM NaCl, 10 mM imidazole). The His-tagged scFv#6 proteins were purified by gravity flow through 1 ml of Ni-NTA agarose (Qiagen), followed by a wash with 20 ml of wash buffer (50 mM Na phosphate, pH 8.5, 300 mM NaCl, 20 mM imidazole) and eluted in 5 ml of elution buffer (50 mM Na phosphate, pH 8.5, 300 mM NaCl, 250 mM imidazole). The eluted protein was subsequently dialysed against 0.1M MES pH6.0, snap frozen and stored at −80° C. at a concentration of 1.4 mg/ml

### Magnetic Particles

Biodegradable magnetic nanoparticles with an average size of 100 nm (by dynamic light scattering) were purchased from Chemicell. Berlin. Germany. These particles are referred to as nano-screenMAG particles with an extension to their name-ARA, referring to the surface coating of the iron oxide core with Glucuronic acid used for the binding to biomolecules, and are covered by a lipophilic fluorescence dye emitting light in the range of green (Exc.=476 nm/Emₘₐₓ.=490 nm).

### Coupling of Fluorescent Magnetic Particles to Proteins

FITC labelled Tat cell penetrating peptide 5(6)-Carboxyfluorescein-GRKKRRQRRRPQ and Gaussia Luciferase secretory peptide 5(6)-Carboxyfluorescein-MGVKVLFALICIAVAEA were synthesised by JPT Peptide Technologies GmbH. The lyophilised peptides were resuspended in 100 μl of DMSO and subsequently diluted down to a concentration of 1 mg/ml with 10 mM PBS pH7.4, snap frozen and stored at −80° C. in aliquots.

The magnetic particles were activated by 2 washes with 1 ml 0.1M MES buffer pH5.0 using the MagnetoPure (Chemicell, Berlin, Germany) magnetic separator. After the second wash the particles were resuspended in 0.25 ml 0.1M MES buffer pH5.0. The carboxyl groups on the green fluorescent magnetic beads nano-screenMAG/G-ARA were activated with freshly prepared EDC (1-Ethyl-3-(3-dimethylaminopropyl)carbodiimide) (Sigma) by dissolving 10 mg EDC in 0.25 ml 0.1M MES buffer. The EDC buffer was added to the particles and gently mixed at RT for 10 min. Following this incubation, the magnetic particles were washed 2× with 1 ml 0.1M MES buffer pH5.0 and resuspended in 0.25 ml of 0.1M MES buffer, pH5.0.

200 μg of scFv#6 purified protein were mixed with 150 μg of Tat cell penetrating peptide and 200 μg of Gaussia luciferase export peptide in 0.25 ml of 0.1M MES buffer, pH 5.0. The protein mixture was added to 10 mg of activated particles and gently mixed for two hours at room temperature to generate magnetic-protein complexes. The particles were washed 3× with 1 ml PBS and resuspended in 500 μl Blocking/Storage buffer (10 mM PBS pH7.4, 0.1% BSA, 0.05% sodium azide).

### Cell Culture

Chinese Hamster Ovary (CHO) cells and the A549 human lung carcinoma cell line (which constitutively express mutant K-RasG12V antigen) were maintained in selective Gibco® Ham's F12 Nutrient Mixture Media (Life Technologies) supplemented with 10% heat-inactivated foetal bovine serum, 100 μg/mL streptomycin, 0.29 mg of L-glutamine at 37° C. in a humidified atmosphere of 5% CO₂.

### Magnetic Intracellular Cell Separation Technique (MICELS)

The CHO and A549 cell lines were seeded the day before the magnetic selection, at 2×10⁵ cells/well in a 24 well glass bottom plate (In Vitro Scientific) in 1 ml of growth media to allow the cells reach 90-95% confluency at the time of the magnetic cell separation. The following day the media was replaced with fresh growth media pre-warmed at 37° C. A serial dilution ranging between 40 μg and 2.4 μg of the magnetic-protein mix was gently mixed with 100 μl of serum free Ham's F12 media and added drop wise to the cells. The cells were incubated with the magnetic-protein complexes for 1.30 h at 37° C. in a humidified atmosphere of 5% CO₂ to allow these to transduce into the cells. The cells were then vigorously washed five times with PBS and incubated further for 3 h. Following the 3 hour incubation, any unbound magnetic-protein complexes are secreted from the cells through the cell secretory pathway. The cells were then vigorously washed five times with PBS and trypsinised in the 24 well plate with 0.05% trypsin-EDTA (Invitrogen). 0.5 ml of fresh F12 growth media was added to each well to inactivate the Trypsin and the 24 well plate was placed on top of a MagnetoPURE (Chemicell, Berlin, Germany) magnetic separator for cell selection. At this point any cells that did not contain magnetic-protein complexes were gently washed away 3 times with 500 μl PBS on the magnetic separator. The magnetic separator was then removed and the remaining cells containing the magnetic-scFv complexes bound to the activated form of Ras were further incubated in 500 μl of growth media at 37° C. in a humidified atmosphere of 5% CO₂ for 48 h.

### Image Acquisition Laser Scanning Confocal Microscopy.

Laser scanning confocal microscopy was carried out using an Inverted Zeiss LSM 510 META Axiovert 200M confocal microscope using a 10×, 20× and a 40×/1.3NA oil immersion objective. FITC was imaged using an Argon 488-nm laser light and a 505-530-nm BP emission filter

### EXAMPLES

A method to separate whole live cells using intracellular proteins rather than cell surface proteins (MICELS-magnetic intracellular cell separation) was developed by coupling magnetic particles to scFv antibody fragments and to cell penetrating and cell export peptides (FIG. 1). In this study the proto-oncogene mutant RasG12V was used as a model system to test the MICELS technology. The Ras protein belongs to a class of proteins known as small GTPases, and plays key roles in signal transduction as molecular switches. These proteins are mediated through two switch regions displaying conformational differences between active (GTP bound) and inactive (GDP bound) states (Vetter and Wittinghofer, 2001). Ocassionally, mutations occur in these Ras proteins (K, H or NRAS) resulting in constitutively activated GTP-bound forms that promote cell transformation in a signal-independent manner (Adjei, 2001), thereby, promoting cell proliferation and protection from apoptosis. Approximately 30% of human cancers contain oncogenic Ras mutants, with higher frequencies in pancreas, colon and lung adenocarcinoma (Grewal et al., 2011). The Ras isoforms (H-Ras, N-Ras and K-Ras) are mainly localised to the inner cell membrane, however, in recent years they have been shown to associate, as well, with intracellular organelles such as the Golgi, the ER, endosomes and the mitochondria (Grewal et al., 2011).

An scFv antibody fragment (scFv#6) previously shown to bind specifically to the activated form of Ras, rather than to particular RAS mutants (Tanaka et al., EMBO Journal 2007), was employed for this study. The gene was synthesised by GeneArt (Life Technologies) and subsequently subcloned into the bacterial expression system pRSET to generate His-Tagged scFv. The protein was expressed in BL21 (DE3) bacterial cells and coupled to green fluorescent magnetic beads together with FITC labeled Tat penetrating peptide to facilitate the transport of the complexes into cells. A FITC labeled Gaussia luciferase export peptide was coupled, as well, to generate the MICELS complexes. The export peptide would direct the magnetic complexes to the extracellular milieu by either following the classical or an unconventional cell secretory pathway (Nickel, 2005). Any complexes that had not anchored to the activated Ras proteins, would be secreted.

A serial dilution of the magnetic-protein complexes ranging from 40 μg and 2.4 μg were incubated with the CHO and A549 cells in a 24 well plate for 1.30 h to allow them to transduce into the cells. The cells were then extensively washed with PBS to remove any complexes that had not transduced into the cells. These were further incubated for another 3 h to allow for the export peptide to re-localise the complexes in the cells thereby allowing hem to exit via the cell export system. This was to ensure the removal of any complexes that had not recognised the Ras antigen. The cells were then vigorously washed again before trypsinisation and selection with the magnet (FIG. 2).

The cells containing the magnetic-protein complexes slowly migrated towards the magnet. These were gently washed with PBS by magnetic separation to ensure the removal of cells that did not contain the magnetic-protein complexes. The supernatant was removed and whole live cells containing activated Ras were selected using the MICELS method and placed back into culture (FIG. 3-A,B,C,D). 24 h later the cells had divided diluting down the cells in culture that still contained the magnetic-protein complex (FIG. 3-E,F). The lipophilic fluorescent dye and the polysaccharid Glucuronic acid coating of the magnetic-protein complexes eventually degrade inside the cell leaving just the iron oxide core of the magnetic bead, which is non-toxic to the cells. It is thought that the iron oxide nanoparticles reside inside lysosomes (Becker et al., 2007) and are eventually degraded into iron ions via enzyme hydrolysis (Shuayev et al., 2009).

### The Magnetic-Protein Complexes Localise to Subcellular Compartments.

Depending on the cell signaling events undergoing in the cell, the H-Ras, N-Ras and K-Ras isoforms have all been shown to localise to the cell membrane as well as to a number of other subcellular compartments such as the ER, the Golgi, endosomes and to the perinuclear membrane (Grewal et al., 2011). In this study the scFv magnetic-protein complexes localised to the cell membrane, to the perinuclear membrane and to distinct subcellular vesicles in the cytoplasm consistent with the localisation pattern of activated Ras in the A549 lung cancer cell line. Optimal results for cell selection were obtained by using 40 μg of complexes. The number of cells selected decreased concomitantly with the decrease in the concentration of complexes (FIG. 2-A).

A simple method is described here for selection of cells using intracellular proteins by coupling antigen-specific antibody fragments to magnetic beads and by using cell penetrating peptides to facilitate the cellular uptake of these complexes, and cell export peptides to allow the secretion of any unbound complexes via the cell secretory pathway.

The method is fast and simple, and requires minimal work. Furthermore, no cellular cytotoxicity was observed allowing the cells to be used for other downstream applications.

### REFERENCES

Adjei A A (2001) Blocking oncogenic Ras signaling for cancer therapy. J Natl Cancer Inst 93: 1062-1074.
AlHajj M, Wicha M S, BenitoHernandez A, Morrison S J, Clarke M F: Prospective identification of tumorigenic breast cancer cells. Proc Natl Acad Sci USA 100: 39833988, 2003.
Alexiou, C., Arnold, W., Klein, R. J., Parak, F. G., Hulin, P., Bergemann, C., Erhardt, W., Wagenpfeil, S., Lubbe, A. S., Locoregional cancer treatment with magnetic drug targeting. Cancer Res. 60:6641-6648, 2000.
Avignolo C., Bagnasco L., Biasotti B., Melchiori A., Tomati V., Bauer I., Salis A., Chiossone L., Mingari M. C., Orecchia P., Carnemolla B., Neri D., Zardi L., Parodi S. Internalization via Antennapedia protein transduction domain of an scFv antibody toward c-Myc protein. The FASEB Journal 22: 1237-1245, 2008.
Becker C., Hodenius M., Blendinger G., Sechi A., Hieronymus T., Müller-Schulte D., Schmitz-Rode T., Zenke M. Uptake of magnetic nanoparticles into cells for cell tracking. J Magn Magn Mater 311:234-237, 2007.
Bigner D. D., Brown M. T., Friedman A. H., Coleman R. E., Akabani G., Friedman H. S., Thorstad W. L., McLendon R. E., Bigner S. H., Zhao X. G., Pegram C. N., Wikstrand C. J., Herndon J. N., Vick N. A., Paleologos N., Cokgor I., Provenzale J. M., Zalutsky M. R. Iodine-131-labeled antitenascin monoclonal antibody 81C6 treatment of patients with recurrent malignant gliomas: Phase I trial results. J. Clin. Oncol., 16: 2202-2212, 1998.
Bonnet D, Dick J. E. Human acute myeloid leukemia is organized as a hierarchy that originates from a primitive hematopoietic cell. Nat. Med. 3: 730737, 1997.
Brinkmann U., Pai L. H., FitzGerald D. J., Willingham M., Pastan I. B3(Fv)-PE38 KDEL, a single-chain immunotoxin that causes complete regression of a human carcinoma in mice. Proc. Natl. Acad. Sci. USA, 88: 8616-8620, 1991.

Brown M. T., Coleman E., Friedman A. H., Friedman H. S., McLendon R. E., Reiman R., Felsberg G. J., Tien R. D., Bigner S. H., Zalutsky M. R., Zhao X. G., Wikstrand C. J., Pegram C. N., Herndon J. N., Vick N. A., Paleologos N., Fredericks R. K., Schold S., Jr., Bigner D. D. Intrathecal ¹³¹I-labeled antitenascin monoclonal antibody 81C6 treatment of patients with leptomeningeal neoplasms or primary brain tumor resection cavities with subarachnoid communication: Phase I trial results. Clin. Cancer Res., 2: 963-972, 1996.
Chester K. A., Begent R. H., Robson L., Keep P., Pedley R. B., Boden J. A., Boxer G., Green A., Winter G., Cochet O., Hawkins R. E. Phage libraries for generation of clinically useful antibodies. Lancet, 343: 455-456, 1994.
Cinti C., Taranta M., Naldi I., Grimaldi S, Newly Engineered Magnetic Erythrocytes for Sustained and Targeted Delivery of Anti-Cancer Therapeutic Compounds. PLoS ONE 6(2): 1-9, 2011.
Collins A T, Berry P A, Hyde C, Stower M J, Maitland N J: Prospective Identification of Tumorigenic Prostate Cancer Stem Cells. Cancer Res. 65: 1094610951, 2005.
Futaki S., Suzuki T., Ohashi W., Yagami T., Tanaka S., Ueda K., Sugiura Y. Arginine-rich peptides. An abundant source of membrane-permeable peptides having potential as carriers for intracellular protein delivery. J Biol chem. 276 (8): 5836-5840, 2001
Grewal T., Koese M., Tebar F., Enrich C. Differential Regulation of RasGAPs in Cancer. Genes & Cancer 2(3) 288-297, 2011
Hamburger A W, Salmon S E: Primary bioassay of human tumor stem cells. Science 197: 461463, 1977
Han Y., Haun Y., Deng J., Gao F., Pan B., Cui D. Expression of single-chain Fv gene specific for gamma-seminoprotein by RTS and its biological activity identification. Biotechnol Prog. 22(4):1084-9, 2006.
Houghton J, Stoicov C, Nomura S, Rogers A B, Carlson J, Li H, Cai X, Fox J G, Goldenring J R, Wang T C: Gastric cancer originating from bone marrow derived cells. Science 306: 15681571, 2004.
Huston J. S., Levinson D., Mudgett-Hunter M., Tai M. S., Novotny J., Margolies M. N., Ridge R. J., Bruccoleri E. H., Crea R., Oppermann H. Protein engineering of antibody binding sites: recovery of specific activity in an anti-digoxigenin single-chain Fv analogue produced in Escherichia coli. Proc. Natl. Acad. Sci. USA, 85:5879-5883, 1988.
Huston J. S., George A. J., Adams G. P., Stafford W. F., Jamar F., Tai M. S., McCartney J. E., Oppermann H., Heelan B. T., Peters A. M., Houston L. L., Bookman M. A., Wolf E. J., Weiner L. M. Single-chain Fv radioimmunotargeting. Q. J. Nucl. Med., 40: 320-333, 1996.
Jain R., Baxter L. Mechanisms of heterogeneous distribution of monoclonal antibodies and other macromolecules in tumors: significance of elevated interstitial pressure. Cancer Res. 48: 7022-7033, 1988.
Jeong J-G, Kim D-S, Kim Y—S, Kwon M-H. A Tat-grafted anti-nucleic acid antibody acquires nuclear-localization property and a preference for TAR RNA. Biochem. Biophys. Res. Commun. 406: 403-40, 2011.
Kaminski M. S., Zasadny K. R., Francis I. R., Milik A. W., Ross C. W., Moon S. D., Crawford S. M., Burgess J. M., Petry N. A., Butchko G. M., Glenn S. D., Wahl R. L. Radioimmunotherapy of B-cell lymphoma with [¹³¹I]anti-B1 (anti-CD20) antibody. N. Engl. J. Med., 329: 459-465, 1993.
Kaminski M. S., Zasadny K. R., Francis I. R., Fenner M. C., Ross C. W., Milik A. W., Estes J., Tuck M., Regan D., Fisher S., Glenn S. D., Wahl R. L. Iodine-131-anti-B1 radioimmunotherapy for B-cell lymphoma. J. Clin. Oncol., 14: 1974-1981, 1996.
Kim C F, Jackson E L, Woolfenden A E, Lawrence S, Babar I., Vogel S, Crowley D, Bronson R T, Jacks T: Identification of bronchioalveolar stem cells in normal lung and lung cancer. Cell 121: 823-835, 2005.
Krotz, F., Wit, C., Sohn, H. Y., Zahler, S., Gloe, T., Pohl, U., Plank, C. Magnetofection-A highly efficient tool for antisense oligonucleotide delivery in vitro and in vivo. Mol. Ther. 7:700-710, 2003.
Laske D. W., Youle R. J., Oldfield E. H. Tumor regression with regional distribution of the targeted toxin TF-CRM107 in patients with malignant brain tumors. Nat. Med., 3: 1362-1368, 1997.
Lubbe, A. S., Bergemann, C., Riess, H., Schriever, F., Reichardt, P., Possinger, K., Matthias, M., Dorken, B., Herrmann, F., Gurtler, R., Hohenberger, P., Haas, N., Sohr, R., Sander, B., Lemke, A. J., Ohlendorf, D., Huhnt, W., Huhn, D. Clinical experiences with magnetic drug targeting: a phase I study with 4′-epidoxorubicin in 14 patients with advanced solid tumors. Cancer Res. 56:4686-4693, 1996.
Ma D X, Shi N Q, Qi X R. Distinct transduction modes of arginine-rich cell-penetrating peptides for cargo delivery into tumor cells. Int J. Pharm. 2011.
McConnell S J, Dinh T, Le M H, Spinella D G. Biopanning phage display libraries using magnetic beads vs. polystyrene plates. Biotechniques. 26(2):208-10, 1999.
Milenic D. E., Yokota T., Filpula D. R., Finkelman M. A. J., Dodd S. W., Wood J. F., Withlow M., Snoy P., Schlom J. Construction, binding properties, metabolism, and tumor targeting of a single chain Fv derived from the pancarcinoma monoclonal antibody CC49. Cancer Res., 51: 6363-6371, 1991.
Morrison P. F., Laske D. W., Bobo H., Oldfield E. H., Dedrick R. L. High-flow microinfusion: tissue penetration and pharmacodynamics. Am. J. Physiol., 266:R292-R305, 1994.
Nickel W. Unconventional Secretory Routes: Direct Protein Export Across the Plasma Membrane of Mammalian Cells. Traffic 6: 607-614, 2005
Niesner U., Halin C., Lozzi L., Günthert M., Neri P., Wunderli-Allenspach H., Zardi L., and Neri D. Quantitation of the Tumor-Targeting Properties of Antibody Fragments Conjugated to Cell-Permeating HIV-1 TAT Peptides. Bioconjugate Chem., 13 (4): 729-736, 2002.
Nord K, Nord O, Uhlen M, Kelley B, Ljungqvist C, Nygren P A. Recombinant human factor VIII-specific affinity ligands selected from phage-displayed combinatorial libraries of protein A. Eur. J. Biochem. 268:4269-4277, 2001
O'Brien C A, Pollett A, Gallinger S, Dick J E: A human colon cancer cell capable of initiating tumour growth in immunodeficient mice. Nature 445: 106110, 2007.
Pardal R, Clarke M F, Morrison S J: Applying the principles of stem cell biology to cancer. Nat. Rev. Cancer 3: 895902, 2003
Pastan I. H., Archer G. E., McLendon R. E., Friedman H. S., Fuchs H. E., Wang Q-C., Pai L. H., Herndon J., Bigner D. D. Intrathecal administration of a single-chain immunotoxin, LMB-7 [B3(Fv)-PE38], produces cures of carcinomatous meningitis in a rat model. Proc. Natl. Acad. Sci. USA, 92: 2765-2769, 1995.
Press O. W., Eary J. F., Appelbaum F. R., Martin P. J., Badger C. C., Nelp W. B., Glenn S., Butchko G., Fisher D., Porter B., Matthews D. C., Fisher L. D., Bernstein I. D. Radiolabeled-antibody therapy of B-cell lymphoma with autologous bone marrow support. N. Engl. J. Med., 329: 1219-1224, 1993.
Raag R., Whitlow M. Single-chain Fvs. FASEB J., 9: 73-80, 1995.
Reisfeld R. A., Gillies S. D. Recombinant antibody fusion proteins for cancer immunotherapy. Curr. Top. Microbiol. Immunol., 213: 27-53, 1996.
Ricci Vitiani L, Lombardi D G, Pilozzi E, Biffoni M, Todaro M, Peschle C, De Maria R: Identification and expansion of human colon cancer initiating cells. Nature 445: 111115, 2007.
Saiyed Z M, Telang S D, Ramchand C N. Application of magnetic techniques in the field of drug discovery and biomedicine. Biomagn. Res. Technol. 1:1-8, 2003.
Scherer F., Anton M., Schillinger U., Henke J., Bergemann C., Kruger A., Gansbacher B., Plank C. Magnetofection: enhancing and targeting gene delivery by magnetic force in vitro and in vivo. Gene Ther. 9:102-109, 2002.
Shuayev V I., Pisanic T R., Jun S. Magnetic nanoparticles for theragnostics. Adv Drug Deliver Rev 61:467-477, 2009.
Singh S K, Hawkins C, Clarke I D, Squire J A, Bayani J, Hide T, Henkelman R M, Cusimano M D, Dirks P B: Identification of human brain tumour initiating cells. Nature 432: 396401, 2004.
Skerra A. Bacterial expression of immunoglobulin fragments. Curr. Opin. Immunol., 5: 256-262, 1993.
Smith C A, de la Fuente J, Pelaz B, Furlani E P, Mullin M, Berry C C. The effect of static magnetic fields and tat peptides on cellular and nuclear uptake of magnetic nanoparticles. Biomaterials 31(15):4392-400, 2010.
Stark D D, Weissleder R, Elizondo G, Hahn P F, Saini S, Todd L E, Wittenberg J and Ferucci J T Superparamagnetic iron oxide: clinical application as a contrast agent for MR imaging of the liver. Radiology 168: 297-301, 1988.
Suzuki M, Honda H, Kobayashi T, Wakabayashi T, Yoshida J and Takahashi M Development of a target directed magnetic resonance contrast agent using monoclonal antibody-conjugated magnetic particles Brain Tumor Pathol. 13:127-32, 1996.
Takkinen K., Laukkanen M. L., Sizmann D., Alfthan K., Immonen T., Vanne L., Kaartinen M., Knowles J. K., Teeri T. T. An active single-chain antibody containing a cellulase linker domain is secreted by Escherichia coli. Prot. Eng., 4: 837-841, 1991.
Tanaka T, Williams R L, Rabbitts T H (2007) Tumour prevention by a single antibody domain targeting the interaction of signal transduction proteins with RAS. EMBO J. 26(13): 3250-3259
Tseng P., Carlo D., Judy J. W. Rapid and Dynamic Intracellular Patterning of Cell-Internalized Magnetic Fluorescent Nanoparticles. Nano Lett. 2009.
Vetter I R, Wittinghofer A (2001) The guanine nucleotide-binding switch in three dimensions. Science 294: 1299-1304.
Weissleder R, Cheng H C, Bogdanova A and Bogdanov A. Magnetically labeled cells can be detected by MR imaging J. Magn. Reson. Imaging 7:258-63, 1997
Winter G., Griffiths A. D., Hawkins R. E., Hoogenboom H. R. Making antibodies by phage display technology. Annu. Rev. Immunol., 12: 433-455, 1994.
Yokota T., Milenic D. E., Withlow M., Schlom J. Rapid tumor penetration of a single-chain Fv and comparison with other immunoglobulin forms. Cancer Res., 52:3402-3408, 1992.
Yuedong Han, Yi Haun, Jinlan Deng, Feng Gao, Bifeng Pan, Daxiang Cui. Expression of Single-Chain Fv Gene Specific for γ-Seminoprotein by RTS and Its Biological Activity Identification. Biotechnology Progress 22 (4): 1084-1089, 2006

1. A magnetic particle comprising: a ligand that specifically binds an intracellular antigen, a cell membrane penetrating cationic peptide and a peptide comprising an amino acid sequence that is adapted to interact with the secretory pathway in a cell.

2. The magnetic particle according to claim 1, wherein said intracellular antigen is a nuclear antigen.

3. The magnetic particle according to claim 2, wherein said nuclear antigen is selected from the group consisting of: FoxO family members, TARP, p53, Rb, E2F, hK4, BRCA1, Cdk2, SATB1, TP53INP1, Myc, Fos, and Jun.

4. The magnetic particle according to claim 1, wherein said intracellular antigen is substantially a cytoplasmic antigen.

5. The magnetic particle according to claim 4, wherein said cytoplasmic antigen is selected from the group consisting of: Jak3, GATA 1-4, Stats 1-6, CBP, NFkB, IKK, PIK3CA, BRAF, PIK3CA, EML4-ALK, VEGF, EBI3, eIF2α, Akt, PI3K, IAP, Hs, Hsp, FAK, Raf, Ras, TNF, Src, Wnt, and Abl.

6. The magnetic particle according to claim 1, wherein said cell membrane penetrating cationic peptide is a natural cell penetrating peptide.

7. The magnetic particle according to claim 1, wherein said cell membrane penetrating cationic peptide is a synthetic sequence.

8. The magnetic particle according to claim 1 wherein said cell membrane penetrating cationic peptide is adapted to penetrate a mammalian cell.

9. The magnetic particle according to claim 1 wherein said cell membrane penetrating cationic peptide is selected from the group consisting of: YARKKRRQRRR (SEQ ID NO: 1), YARKARRQARR (SEQ ID NO: 2), YARAAARQARA (SEQ ID NO: 3), YARAARRAARR (SEQ ID NO: 4), YARAARRAARA (SEQ ID NO: 5), YARRRRRRRRR (SEQ ID NO: 6), YAAARRRRRRR (SEQ ID NO: 7), PLSSIFSRIGDP (SEQ ID NO: 8), RQIKIWFQNRRMKWKK (SEQ ID NO: 9), WEIEDEDER (SEQ ID NO: 10), GRKKRRQRRRPQ (SEQ ID NO: 11), and RRRRRRRRRRRR (SEQ ID NO: 12).

10. The magnetic particle according to claim 9 wherein said cell membrane penetrating cationic peptide comprises the amino acid sequence: GRKKRRQRRRPQ (SEQ ID NO: 11).

11. (canceled)

12. The magnetic particle according to claim 9, wherein said cell membrane penetrating cationic peptide comprises the amino acid sequence: RRRRRRRRRRRR (SEQ ID NO: 12).

13. (canceled)

14. The magnetic particle according to claim 1, wherein said peptide adapted to interact with the mammalian secretory pathway is selected from the group consisting of: MCPARSLLLVATLVLLDHLSLA (SEQ ID NO: 13), MDAMKRGLCCVLLLCGAVFVSPS (SEQ ID NO: 14), MATGSRTSLLLAFGLLCLPWLQEGSA (SEQ ID NO: 15), MYRMQLLSCIALSLALVTNS (SEQ ID NO: 16) and MGVKVLFALICIAVAE (SEQ ID NO: 17).

15. The magnetic particle according to claim 12 wherein said peptide comprises the amino acid sequence MGVKVLFALICIAVAE (SEQ ID NO: 17).

16. (canceled)

17. The magnetic particle according to claim 12 wherein said peptide comprises the amino acid sequence: MYRMQLLSCIALSLALVTNS (SEQ ID NO: 16).

18. (canceled)

19. The magnetic particle according to claim 1 wherein said ligand that binds said intracellular antigen is an antibody fragment.

20. The magnetic particle according to claim 1 wherein said antibody fragment is a ScFv.

21. The magnetic particle according to claim 1 wherein said ligand that binds said intracellular antigen is a peptide or a peptide aptamer.

22-23. (canceled)

24. The magnetic particle according to claim 1 wherein said magnetic particle further comprises a nuclear export peptide.

25. The magnetic particle according to claim 18 wherein said nuclear export peptide comprises the amino acid sequence: NELALKLAGLDINKTEGEEDAQ (SEQ ID NO: 18).

26. A method for the selection and isolation of a mammalian cell from a heterogeneous population of cells comprising:
i) forming a first preparation comprising a heterogeneous population of mammalian cells;
ii) contacting said population with a second preparation comprising a magnetic particle according to claim 1 to provide a combined preparation and providing conditions that allow penetration and binding of said magnetic particle to an intracellular antigen;
iii) incubating said combined preparation to remove unbound magnetic particles by the secretory pathway of said mammalian cell, optionally by application of a magnetic field; and
iv) applying a magnetic field to said combined preparation to select and isolate mammalian cells comprising magnetic particles bound to an intracellular antigen.
v)

27. The method according to claim 20 wherein said mammalian cell is a stem cell.

28. The method according to claim 25 wherein said stem cell is a cancer stem cell.

29. A kit for customized modification of magnetic particles, comprising:
containing a selection of cell membrane penetrating cationic peptides selected from the group of YARKKRRQRRR (SEQ ID NO: 1), YARKARRQARR (SEQ ID NO: 2), YARAAARQARA (SEQ ID NO: 3), YARAARRAARR (SEQ ID NO: 4), YARAARRAARA (SEQ ID NO: 5), YARRRRRRRRR ((SEQ ID NO: 6), YAAARRRRRRR (SEQ ID NO: 7), PLSSIFSRIGDP (SEQ ID NO: 8), RQIKIWFQNRRMKWKK (SEQ ID NO: 9), WEIEDEDER (SEQ ID NO: 10), GRKKRRQRRRPQ (SEQ ID NO: 11), and RRRRRRRRRRRR (SEQ ID NO: 12);
a selection of secretory proteins selected from the group MCPARSLLLVATLVLLDHLSLA (SEQ ID NO: 13), MDAMKRGLCCVLLLCGAVFVSPS (SEQ ID NO: 14), MATGSRTSLLLAFGLLCLPWLQEGSA (SEQ ID NO: 15), MYRMQLLSCIALSLALVTNS (SEQ ID NO: 16) and MGVKVLFALICIAVAE (SEQ ID NO: 17);
activation, wash and blocking/storage buffer; and
optionally a nuclear export peptide.

30-32. (canceled)


SEQUENCE LISTING (18 sequences)

SEQ ID NO: 1 - protein, 11 aa, Artificial Sequence
  cell membrane penetrating peptide
YARKKRRQRRR

SEQ ID NO: 2 - protein, 11 aa, Artificial Sequence
  cell membrane penetrating peptide
YARKARRQARR

SEQ ID NO: 3 - protein, 11 aa, Artificial Sequence
  cell membrane penetrating peptide
YARAAARQARA

SEQ ID NO: 4 - protein, 11 aa, Artificial Sequence
  cell membrane penetrating peptide
YARAARRAARR

SEQ ID NO: 5 - protein, 11 aa, Artificial Sequence
  cell membrane penetrating peptide
YARAARRAARA

SEQ ID NO: 6 - protein, 11 aa, Artificial Sequence
  cell membrane penetrating peptide
YARRRRRRRRR

SEQ ID NO: 7 - protein, 11 aa, Artificial Sequence
  cell membrane penetrating peptide
YAAARRRRRRR

SEQ ID NO: 8 - protein, 12 aa, Artificial Sequence
  cell membrane penetrating peptide
PLSSIFSRIGDP

SEQ ID NO: 9 - protein, 16 aa, Artificial Sequence
  cell membrane penetrating peptide
RQIKIWFQNRRMKWKK

SEQ ID NO: 10 - protein, 9 aa, Artificial Sequence
  Cell membrane penetrating peptide
WEIEDEDER

SEQ ID NO: 11 - protein, 12 aa, Artificial Sequence
  cell membrane penetrating peptide
GRKKRRQRRRPQ

SEQ ID NO: 12 - protein, 12 aa, Artificial Sequence
  cell membrane penetrating peptide
RRRRRRRRRRRR

SEQ ID NO: 13 - protein, 22 aa, Artificial Sequence
  secretory peptide
MCPARSLLLVATLVLLDHLSLA

SEQ ID NO: 14 - protein, 23 aa, Artificial Sequence
  secretory peptide
MDAMKRGLCCVLLLCGAVFVSPS

SEQ ID NO: 15 - protein, 26 aa, Artificial Sequence
  secretory peptide
MATGSRTSLLLAFGLLCLPWLQEGSA

SEQ ID NO: 16 - protein, 20 aa, Artificial Sequence
  secretory peptide
MYRMQLLSCIALSLALVTNS

SEQ ID NO: 17 - protein, 16 aa, Artificial Sequence
  secretory peptide
MGVKVLFALICIAVAE

SEQ ID NO: 18 - protein, 22 aa, Artificial Sequence
  Nuclear export peptide
NELALKLAGLDINKTEGEEDAQ